(19) United States
(12) Patent Application Publication
(10) Pub. No.: US 2002/0168431 A1
(43) Pub. Date: Nov. 14, 2002

(54) Methods for the treatment and prevention of metabolic diseases including diabetes

(76) Inventor: David Belna, Costa Mesa, CA (US)

Correspondence Address: Ray K. Shahani, Esq., Twin Oaks Office Plaza, Suite 112, 477 Ninth Avenue, San Mateo, CA, 94402-1854, US

(21) Appl. No.: 10/093,949

(22) Filed: Mar. 7, 2002

Related U.S. Application Data
(60) Provisional application No. 60/274,485, filed on Mar. 7, 2001.

(51) Int. Cl. A61K 35/78

(52) U.S. Cl. 424/746

(57) ABSTRACT

A method of treating metabolic diseases such as diabetes, hypoglycemia, or hypoglycemia in a patient includes providing Chia seeds, having a seed body, adding water to the Chia seeds to form a gel, separating the seed body from the gel, extracting a water soluble fiber including a polysaccharide from the gel, and delivering a therapeutic amount of the fiber to the digestive tract of the patient. The step of delivering a therapeutic amount can include combining the fiber and polysaccharide with a carbohydrate-based food. The method can further include adding a bio-adhesive, and/or an amylase inhibitor to the gel, or the polysaccharide.

### RELATED INVENTIONS

This Application is related to and incorporates herein by reference, in its entirety, U.S. Provisional Application Serial No. 60/274,485 filed Mar. 7, 2001 entitled METHODS FOR THE TREATMENT AND PREVENTION OF METABOLIC DISEASES INCLUDING DIABETES, and claims any and all benefits to which it is entitled therefrom.

### FIELD OF THE INVENTION

The present invention relates to the treatment of metabolic diseases including diabetes mellitus (diabetes), and particularly to methods of optimizing the digestion of carbohydrates in humans.

### BACKGROUND OF THE INVENTION

Research reveals that over half of all deaths in U.S. are diet-related, that is, the cause of death has been identified as having statistical correlation to a particular dietary scheme. Metabolic diseases including cardiovascular disease and diabetes mellitus had been statistically linked to dietary habits. Cardiovascular disease is the top killer. Cancer is second. Diabetes and its complications are the third leading cause of death and Canada and U.S.

According to a report issued by the National Commission on Diabetes, as many as 10 million North Americans may have diabetes, and new incidents are increasing yearly. Although the acute and often lethal symptoms of diabetes can be controlled by insulin therapy, the long-term complications may reduce life expectancy, as much as ⅓. For example, the incidence for blindness is increased by five fold in diabetic patients. Similarly, the incidence is increased seventeen-fold for kidney disease, five fold for gangrene, and two-fold for heart disease.

There are two major forms of diabetes, Type I diabetes, which is also known as insulin dependent diabetes mellitus, and Type II diabetes. Most patients with Type I diabetes have a common pathological feature: the nearly total disappearance of insulin producing pancreatic data cells which results in hyperglycemia.

Type II diabetes, also known as non insulin dependent diabetes, is more common than type I diabetes, affecting 90 percent of all persons with diabetes. Type II diabetes is a mild form of diabetes, and often of gradual onset, which usually appears in middle age. Fortunately, diet alone can be effective to control the abnormal blood glucose levels as seen in Type I diabetes.

A high protein/low carbohydrate diet, for example, has been used to regulate Type II diabetes. Unfortunately, not all patients are willing to radically change their diets and significantly cut their carbohydrate intake. Also, there are long-term health risks associated with high protein diet including health risks linked with excessive intake of meat products, which may to contribute to the onset of coronary and other types of disease.

U.S. Pat. No. 4,801,582 discloses a method for treating hypoglycemia using aloe polysaccharides. This U.S. Patent is incorporated herein by reference. While the aloe polysaccharides appear somewhat effective at moderating plasma glucose levels, there is still in need for improved methods of treating hypoglycemia and other metabolic diseases.

Effective ways to treat and prevent diabetes, and other metabolic disease are desired. Improved ways to moderate the digestion of carbohydrates are also desired. What is further desired is a way of preventing or treating hypoglycemia, hyperglycemia and other metabolic disorders while achieving proper nutrition.

### SUMMARY AND ADVANTAGES

A method of treating metabolic diseases such as diabetes, obesity, hyperglycemia, or hypoglycemia in a patient includes providing Chia seeds having a seed body, adding water to the Chia seeds to form a gel, separating the seed body from the gel, extracting a polysaccharide from the gel, and delivering a therapeutic amount of the polysaccharide to the digestive tract of the patient.

The step of separating the seed body from the gel, according to one aspect of the invention, is accomplished by rinsing the seed with a volume of water roughly twice the seed volume at low temperature (preferably less than 65 degrees Fahrenheit) for a short period (preferably less than 60 seconds). This separates water soluble fiber from the seed without causing an undue amount of gel to form on the seeds. Collecting the rinse water isolates the water soluble fiber, enabling the water soluble fiber to gel without contact with the seed. Repeating the step of rinsing optimizes the yield and, in the proportions specified, enables the gel to fully form in the rinse water.

A further step includes reducing the gel by drying the gel in a vacuum at low temperature (less than 200 degrees Fahrenheit, an preferably less than 100 degrees Fahrenheit). The polysaccharide can be derived from the dried gel, or from the gel directly.

The step of delivering a therapeutic amount can include combining the polysaccharide with a carbohydrate-based food or a means to enhance binding of the polysaccharide to a mucosal membrane within the digestive tract. The method can further include adding a bio-adhesive, and/or an amylase inhibitor to the gel, or the polysaccharide compound.

### BRIEF DESCRIPTION OF THE DRAWINGS

FIG. 1 shows a method of treating a patient for hyperglycemia, hypoglycemia, obesity or diabetes and providing for overall improvements in nutritional intake, digestion and assimilation of foods.

### DETAILED DESCRIPTION OF THE PREFERRED EMBODIMENT

The description that follows is presented to enable one skilled in the art to make and use the present invention, and is provided in the context of a particular application and its requirements. Various modifications to the disclosed embodiments will be apparent to those skilled in the art, and the general principals discussed below may be applied to other embodiments and applications without departing from the scope and spirit of the invention. Therefore, the invention is not intended to be limited to the embodiments disclosed, but the invention is to be given the largest possible scope which is consistent with the principals and features described herein.

It will be understood that in the event parts of different embodiments have similar functions or uses, they may have been given similar or identical reference numerals and descriptions. It will be understood that such duplication of reference numerals is intended solely for efficiency and ease of understanding the present invention, and are not to be construed as limiting in any way, or as implying that the various embodiments themselves are identical.

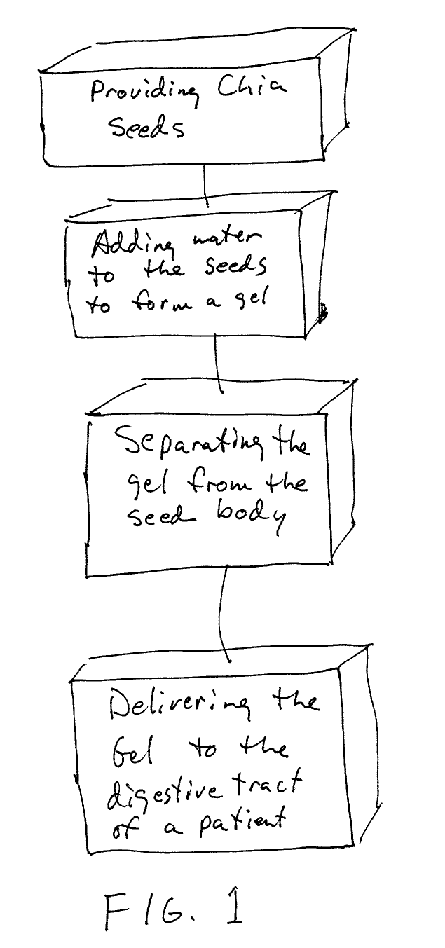

FIG. 1 shows a method of treating a patient for hyperglycemia, hypoglycemia, obesity or diabetes and providing for overall improvements in nutritional intake, digestion and assimilation of foods. The method is based on compounds, mixtures and materials derived from the naturally occurring Chia seed, or synthetic derivatives thereof. These substances regulate the digestion of carbohydrate to achieve improved nutrition and to maintain healthy blood glucose levels.

The Chia plant is from the family labiatae, commonly referred to as Mint. There are two particular types of Chia plants, columbaraie, and salvia hispanica. The columbaraie plants are indigenous to the Southwestern U.S., and grow in the desert climate. The salvia hispanica plant is native to southern Mexico and Guatemala.

One therapeutic method includes administering to a patient afflicted with diabetes, hyperglycemia, or hypoglycemia a therapeutically effective amount of gel formed from the Chia seed. The Chia seed gel is formed by adding water to the seeds, stirring, and allowing soluble fiber from the seeds to gel. Normally, 7-12 parts water to one part Chia seed (by volume) provides sufficient hydration to form the Chia seed gel.

A composition in accordance with the present invention is achieved by dehydrating the Chia seed gel into a powder, resulting in one, or a mixture of, polysaccharides in a solid form. This solid can be delivered as such, or modified to form a liquid or gel. The composition is therapeutically delivered orally, in the form of a powder, tablets including sublingual tablets, emulsion, encapsulated preparations, granules, pellets, liquid preparations including extract, syrup, etc.

Examples of useful excipients include powder, granules, or other oral preparation such as lactose, carbohydrate, calcium phosphate, calcium carbonate, synthetic and natural aluminum silicates, magnesium oxide, dry aluminum hydroxide, magnesium stearate, sodium bicarbonate, dry yeasts, etc.

Chia seed gel, it is suspected, contains a particular polysaccharide, or group of polysaccharides, that favorably affect the digestion of carbohydrates in humans. It has been observed that ingestion of the Chia seed gel decreases the rate at which carbohydrates are digested. While the exact mechanism is yet to be fully understood, there are numerous possible explanations for how the Chia seed gel and derivatives work. One possible explanation is that the Chia seed gel coats the linings of the digestive track and physically inhibits the assimilation of carbohydrates, or shields the carbohydrates from digestive enzyme action.

Another explanation is that the Chia seed gel absorbs significance amounts of water, thereby reducing the local concentration of water and decreasing the rate of hydrolysis.

A further explanation is that the Chia seed gel contains particular polysaccharides that entrap or bond with various monosaccharides and disaccharides within the digestive system, thereby inhibiting or slowing the digestion and/or absorption of monosaccharides and disaccharides.

There are other explanations, including amylase inhibition properties of the Chia seed gel. It is also possible that the Chia seed gel inhibits detection of monosaccharides and disaccharides by receptors in the walls of the digestive tract and thereby inhibits secretion of amylase and other digestive enzymes.

According to one aspect of the invention, the method includes providing Chia seeds, having a seed body, adding water to the Chia seeds to form a gel, separating the seed body from the gel; and delivering the gel to the digestive tract of the patient. Separating the seed body from the gel causes the gel to be more palatable and enables the gel to be reduced to a desired compound, or mixture.

One aspect of the invention includes mixing the gel with a carbohydrate-based food and delivering the carbohydrate-based food to the digestive tract of the patient.

Chia seeds are processed in any of a number of ways. According to one aspect of the invention water is added to the seeds so that the gel forms and then the gel is cold pressed to separate the seed body from the gel. Centrifugation is an effective alternative to separate the seed body from the gel.

Adding a solvent to the Chia seed gel effectuates processing of the Chia seeds. The Chia seed gel can be reduced and encapsulated for oral delivery such as desirable when using the gel as a dietary supplement or in accordance with pharmaceutical purposes. A method in accordance with the present invention includes providing Chia seeds, having a seed body, adding water to the Chia seeds to form a gel, separating the seed body from the gel, and extracting a polysaccharide from the gel.

Delivering the extracted polysaccharide to the digestive tract of the patient not only improves digestion but also prevents radical changes in blood glucose levels. Such radical changes are associative with hypoglycemia, hyperglycemia and diabetes.

The method further includes combining the polysaccharide with a carbohydrate-based food. Adjusting the carbohydrate-based food with the selected polysaccharide enables the polysaccharide to function as a bio-adhesive, binding to a mucosal membrane within the digestive tract.

It can be appreciated however that the method could include any of a number of bio-adhesive to the Chia seed gel, or derivative thereof. U.S. Pat. No. 6,123,965, the disclosure of which is incorporated herein by reference, discloses various methods and compositions for enhancing the bio-adhesive properties of polysaccharides.

Preferably, the bio-adhesive includes a metal compound such as a metal compound selected from the group consisting of: calcium oxide; iron oxide; copper oxide; and zinc oxide.

According to another aspect of the invention an amylase inhibitor is added to the Chia seed gel and ingested. A useful amylase inhibitor of wheat origin is disclosed in U.S. Pat. No. 5,332,803, the disclosure of which is incorporated herein by reference. Accordingly, this amylase inhibitor can inhibit the hydrolysis of carbohydrate into glucose thereby moderate increases in blood glucose levels, ultimately minimizing the need for insulin secretions. It can be appreciated that any of a number of natural or synthetic amylase inhibitors can be substituted for the wheat originated amylase inhibitor in accordance with the present invention.

Since the Chia seed is capable of decreasing the rate of carbohydrate digestion/absorption, the present invention further provides a method of preventing or treating obesity comprising providing the Chia seed gel to a patient diagnosed with obesity. The Chia see is provided in gel form, or in seed form, during meals. According to an alternate aspect of this method, the Chia seed is reduced to a pharmaceutical composition and combined with a compatible excipient.

### ADDITIONAL COMPOSITIONS

A composition, in accordance with the present invention includes the steps comprising:

(a) providing Chia seeds, having a seed body;

(b) adding water to the Chia seeds to form a gel;

(c) separating the seed body from the gel; and

(d) collecting the gel.

The seed body is separated from the gel by pressing the gel, or by centrifugation. An amylase inhibitor, or a bio-adhesive, or both can be added to the composition.

The separated gel can be reduced and added to a carbohydrate-based food such as breads; beverages; crackers; potato chips; synthesized snack foods; and drinks.

Under some circumstances separation of useful polysaccharides from the composition is desirable. Accordingly, the steps of extracting at least one polysaccharide from the gel and collecting the polysaccharide are performed.

A pharmaceutical composition useful in the treatment of diabetes, hyperglycemia, hypoglycemia or obesity is derived in accordance with the present invention. A pharmaceutically compatible excipient is added to form the pharmaceutical composition. Further, a pharmaceutically compatible carrier is added. One aspect of the invention of the pharmaceutical composition includes an amylase inhibitor, or a bio-adhesive, or both, added to the composition.

Under various circumstances it is desirable to include the seed body with the composition for nutritional value and for increased fiber content of the composition. Under other circumstances it is desirable to retain all components of the Chia gel including pectin, oils, minerals, complex carbohydrates, and other components. Still, under various instances it is desired to reduce the Chia gel into more fundamental compositions, including a composition including one or more useful polysaccharides. The various listed metabolic diseases can be treated by either of these Chia seed derivatives.

One exemplary composition is prepared by the steps comprising:

(a) providing Chia seeds, having a seed body;

(b) rinsing the Chia seeds with water to form a gel; and

(c) separating the seed body from the gel by collecting the water.

In nature, the gel normally adheres to the Chia seed body after water is added to the seed, and the gel is challenging to thereafter remove. The present method includes quickly rinsing the seeds to wisk incremental portions of gel-forming water soluble fiber from the seeds, before the gel forms and adheres to the seed body. This is best accomplished in cold water as warm water accelerates gel formation.

The method step of separating includes rinsing the seeds with water at having temperature below 65 degrees, and preferably water having a temperature between 40-60 degrees Fahrenheit to inhibit gel formation on the seed body.

The rinsing will occur during a period of less than 90 seconds to inhibit gel formation on the seed body. Optimally the period is between 30-60 seconds to inhibit gel formation on the seed body. Of course, the time for rinsing can be modified in accordance with the water temperature.

According to one aspect of the invention where hot water (greater than room temperature) is used, the rinsing period is very short e.g. less than 30 seconds.

In any event, the rinsing process repeats to optimize yield. According to one aspect of the invention, the total volume of rinse water is approximately ten times the volume of seed. This enables the rinse water to be collected and enables the gel to form in the rinse water without an excess of, or a shortage of, water. The rinsing process optimally repeats between 6-10 times, but this number can be modified according to the temperature of the water and the rinse period length.

A solvent can be added to the seed, or to the rinse water, to prevent the gel from forming on the seed. Solvents may include alcohol, or other known solvent.

The seeds are agitated during the rinsing process. According to one aspect of the invention, the seeds are slowly centrifuged. Alternatively, a shaker shakes the seeds during rinsing.

The composition, if desired to be reduced from a gel, or if a gel has not optimally formed, is dried at low temperature in a vacuum to minimize any loss of efficacy and nutrition. Preferably, the gel or solution including the water soluble fiber is collected in the vacuum and dried at a temperature of less than 200 degrees Fahrenheit.

A method in accordance with the invention includes crushing the seeds before the step of rinsing to optimize the surface area of the seed and to improve the soluble fiber gathering process.

An example of a method that works include preparing a composition prepared by the steps comprising:

(a) providing Chia seeds, having a seed body;

(b) rinsing the Chia seeds with in water having a volume of no more than two times the Chia seed volume;

(c) collecting the water; and

(d) repeating the step (b) and the step (c), whereby gel forms in the collected water.

Delivering this composition to a diabetic, obese, hyperglycemic, or hypoglycemic patient is anticipated.

Though the present invention is described in terms of treating human patients, it should be known that the invention can be practiced to treat animals, particularly mammals. The Chia seed and its derivatives can be used as a food supplement to enhance the diet and regulate the digestion in livestock. Further, the composition and method of the present invention are useful for cosmetic products, and a fat substitute in foods.

Unless defined otherwise, all technical and scientific terms used herein have the same meaning as commonly understood by one of ordinary skill in the art to which the present invention belongs. Although any methods and materials similar or equivalent to those described can be used in the practice or testing of the present invention, the preferred methods and materials are now described. All publications and patent documents referenced in the present invention are incorporated herein by reference.

While the principles of the invention have been made clear in illustrative embodiments, there will be immediately obvious to those skilled in the art many modifications of structure, arrangement, proportions, the elements, materials, and components used in the practice of the invention, and otherwise, which are particularly adapted to specific environments and operative requirements without departing from those principles. The appended claims are intended to cover and embrace any and all such modifications, with the limits only of the true purview, spirit and scope of the invention.

We claim:

1. A method of treating a patient for diabetes, comprising:
providing Chia seeds, having a seed body;
adding water to the Chia seeds to form a gel;
separating the seed body from the gel; and
delivering the gel to the digestive tract of the patient.

2. A method as set forth in claim 1, wherein the step of delivering the gel comprises mixing the gel with a carbohydrate-based food and delivering the carbohydrate-based food to the digestive tract of the patient.

3. A method as set forth in claim 1, further comprising:
purifying the Chia seeds.

4. A method as set forth in claim 1, wherein the seed body is separated from the gel by pressing the gel.

5. A method as set forth in claim 1, wherein the seed body is separated from the gel by centrifugation.

6. A method as set forth in claim 1, wherein the step of separating the seed body from the gel comprises adding a solvent to the gel.

7. A method as set forth in claim 1, further comprising:
encapsulating the gel for oral delivery.

8. A method of treating a patient for obesity, comprising:
providing Chia seeds, having a seed body;
adding water to the Chia seeds to form a gel;
separating the seed body from the gel; and
delivering the gel to the digestive tract of the patient.

9. A method as set forth in claim 8, wherein the step of delivering the gel includes mixing the gel with a carbohydrate-based food and delivering the carbohydrate-based food to the digestive tract of the patient.

10. A method as set forth in claim 8, further comprising:
purifying the Chia seeds.

11. A method as set forth in claim 8, wherein separating includes pressing the seed body from the gel.

12. A method as set forth in claim 8, wherein the seed body is separated from the gel by centrifugation.

13. A method as set forth in claim 8, wherein the step of separating the seed body from the gel comprises adding a solvent to the gel.

14. A method as set forth in claim 8, further comprising:
encapsulating the gel for oral delivery.

15. A method of improving digestion in a patient, comprising:
providing Chia seeds, having a seed body;
adding water to the Chia seeds to form a gel;
separating the seed body from the gel;
extracting a water soluble fiber including a polysaccharide from the gel; and
delivering the polysaccharide to the digestive tract of the patient.

16. A method as set forth in claim 15, further comprising:
combining the polysaccharide with a carbohydrate based food.

17. A method as set forth in claim 15, further comprising:
binding the polysaccharide to a mucosal membrane within the digestive tract.

18. A method as set forth in claim 17, further comprising:
adding a bio-adhesive to the polysaccharide.

19. A method as set forth in claim 18, wherein the bio-adhesive includes a metal compound.

20. A method as set forth in claim 19, where in the metal compound is selected from the group consisting of:
calcium oxide;
iron oxide;
copper oxide; and
zinc oxide.

21. A method as set forth in claim 15, further comprising:
adding an amylase inhibitor to the polysaccharide.

22. A method for treating diabetes, comprising administering to a patient afflicted with diabetes a therapeutically effective amount of a polysaccharide contained in the seed of the family salvia hispanica.

23. A method for treating diabetes, comprising administering to a patient afflicted with diabetes a therapeutically effective amount of a polysaccharide contained in the seed of the family columbaraie.

24. A method for treating diabetes, comprising administering to a patient afflicted with diabetes a therapeutically effective amount of a polysaccharide contained in a seed of the family labiatae.

25. A composition prepared by the steps comprising:
(a) providing Chia seeds, having a seed body;
(b) adding water to the Chia seeds to form a gel;
(c) separating the seed body from the gel; and
(d) collecting the gel.

26. The composition as set forth in claim 25 wherein the seed body is separated from the gel by pressing the gel.

27. The composition as set forth in claim 25 wherein the seed body is separated from the gel by centrifugation.

28. The composition as set forth in claim 25 further comprising a carbohydrate-based food.

29. The composition as set forth in claim 28 wherein the carbohydrate-based food is selected from the group consisting of: breads; beverages; crackers; potato chips; synthesized snack foods; and drinks.

30. The composition as set forth in claim 25 further comprising an amylase inhibitor.

31. The composition as set forth in claim 25 further comprising a bio-adhesive.

32. The composition as set forth in claim 25 wherein the preparation steps further comprising extracting at least one polysaccharide from the gel and collecting the polysaccharide.

33. A pharmaceutical composition useful in the treatment of diabetes, hyperglycemia, hypoglycemia or obesity, comprising the composition as set forth in claim 25 and a pharmaceutically compatible excipient.

34. The pharmaceutical composition as set forth in claim 33, further comprising a pharmaceutically compatible carrier.

35. The pharmaceutical composition as set forth in claim 33, further comprising an amylase inhibitor.

36. The pharmaceutical composition as set forth in claim 33, further comprising a bio-adhesive.

37. A composition prepared by the steps comprising:
(a) providing Chia seeds, having a seed body;
(b) rinsing the Chia seeds with water to form a gel; and
(c) separating the seed body from the gel by collecting the water.

38. A composition as set forth in claim 37, wherein separating includes rinsing the seeds with water at having temperature below 65 degrees Fahrenheit to inhibit gel formation on the seed body.

39. A composition as set forth in claim 37, wherein separating includes rinsing the seeds with water having a temperature below 65 degrees Fahrenheit, for a period of less than 90 seconds to inhibit gel formation on the seed body.

40. A composition as set forth in claim 37, wherein separating includes rinsing the seeds with water having a temperature below 65 degrees Fahrenheit, for a period of between 30-60 seconds to inhibit gel formation on the seed body.

41. A composition as set forth in claim 37, wherein separating includes rinsing the seeds with water having a temperature between 40-60 degrees Fahrenheit, for a period of between 30-60 seconds.

42. A composition as set forth in claim 37, wherein separating includes rinsing the seeds with water having a temperature between 40-60 degrees Fahrenheit, for a period of between 30-60 seconds, and repeating the step of rinsing at least six times.

43. A composition as set forth in claim 37, wherein separating includes rinsing the seeds with water at a temperature between 40-60 degrees Fahrenheit, for a period of between 30-60 seconds, and repeating the step of rinsing between 6-10 times.

44. A composition as set forth in claim 37, wherein separating includes rinsing the seeds with water at a temperature less than 65 degrees Fahrenheit, for a period of between 30-60 seconds, and repeating the step of rinsing between 6-10 times.

45. A composition as set forth in claim 37, further comprising agitating the seeds.

46. A composition as set forth in claim 37, further comprising drying collected gel in a vacuum.

47. A composition as set forth in claim 46, further comprising drying the gel in a vacuum at a temperature of less than 200 degrees Fahrenheit.

48. A composition as set forth in claim 37, further comprising crushing the seeds before the step of rinsing.

49. A composition prepared by the steps comprising:
(a) providing Chia seeds, having a seed body;
(b) rinsing the Chia seeds with in water having a volume of no more than two times the Chia seed volume;
(c) collecting the water; and
(d) repeating the step (b) and the step (c), whereby gel forms in the collected water.

50. A composition as set forth in claim 49, wherein the water has a temperature of less than 60 degrees Fahrenheit.

51. A method of treating a patient for hypoglycemia, or hyperglycemia, comprising:
providing Chia seeds, having a seed body;
adding water to the Chia seeds to form a gel;
separating the seed body from the gel; and
delivering the gel to the digestive tract of the patient.
